(19) United States
(12) Patent Application Publication
(10) Pub. No.: US 2007/0028340 A1
(43) Pub. Date: Feb. 8, 2007

(54) Interchangeable decorative eye patch cover

(76) Inventor: Shoshanna Nicole Israel, Maple Glen, PA (US)

Correspondence Address: Shoshanna N. Israel, 1328 Arthur Ave, Maple Glen, PA, 19002, US

(21) Appl. No.: 11/197,412

(22) Filed: Aug. 5, 2005

(51) Int. Cl. A61F 9/00

(52) U.S. Cl. 2/15

(57) ABSTRACT

The interchangeable decorative eye patch cover is affixed to a commonly found store-bought eye patch by means of a Velcro backing. These inexpensive covers will come in a variety of fabrics and colors and will allow the wearer to coordinate the look of the eye patch from a fashion perspective to the clothes they are wearing. This will alleviate any discomfort the wearer may have to the fact that common eye patches come only in black and as a result usually do not match what the person is wearing.

### FIELD OF INVENTION

The present invention provides an interchangeable decorative eye patch cover which can be used with commonly found store-bought eye patches.

### BACKGROUND OF THE INVENTION

For various medical reasons people may have partial or total loss of vision in one eye (the bad eye) that may occur on a temporary or permanent basis.

In the case of a partial loss of vision, the person may be required to wear an eye patch over the eye with the loss of vision so that the bad eye does not interfere with the ability of the person to view with the good eye.

In the case of total loss of vision, the person may be compelled to wear an eye patch over the eye with the loss of vision because he or she may be uncomfortable having other people look or even stare at the bad eye.

Currently, eye patches come in one color, black, and as a result are usually viewed as unsightly by the wearer. This may result in the wearer not wanting to wear the eye patch and as a result suffering either from (a) headaches or eye strain as their brain tries to sort out the vision from either the good and bad eyes or (b) embarrassment as they observe other people fixating on their bad eye.

I got this idea when my mother who suffers from Multiple Sclerosis had a flair up which affected her optic nerve and left her temporarily with partial loss of vision in one of her eyes. She felt it was necessary to wear the eye patch but did so sparingly. She complained that the patch was unsightly and did not match any of her clothes.

### SUMMARY OF THE INVENTION

What is needed is an interchangeable decorative eye patch cover.

The interchangeable decorative eye patch cover will be affixed on top of commonly found store-bought eye patches. The cover will be of the same size and shape as the patch portion of the store-bought eye patch. On the back of the cover will be a strip of Velcro. This will attach to another strip of Velcro which will be affixed on the store-bought eye patch. The user can than attach the two strips of Velcro allowing the cover to fit over the store-bought eye patch.

The covers will come in different fabrics and colors allowing the user to choose which cover best fits their clothes based on the wearer's distinctive fashion taste.

The covers will be interchangeable. The user can acquire several different covers with distinctive colors and fabrics and have in essence a wardrobe of covers that can be chosen from based on the wearer's desired fashion needs.

### BRIEF DESCRIPTION OF THE DRAWINGS

FIG. 1 is a perspective view of the interchangeable decorative eye patch cover assembled on to a commonly found store-bought eye patch as worn by a user.

The figure illustrates how the interchangeable decorate eye patch cover matches the design of the user's clothes.

FIG. 2 is a perspective view of the interchangeable decorative eye patch cover assembled on to a commonly found store-bought eye patch.

FIG. 3 is a perspective view of the interchangeable decorative eye patch cover as well as the store-bought eye patch. Furthermore, the figure illustrates the Velcro backing of the interchangeable decorative eye patch cover. In addition the figure illustrates the Velcro strip attached on to the store-bought eye patch

FIG. 4 is a sectional view taken along line 4-4 of FIG. 1 showing the interchangeable decorative eye patch cover assembled on to the store-bought eye patch via the Velcro strip.

FIG. 5 is a perspective view of the store-bought eye patch with the Velcro strip attached.

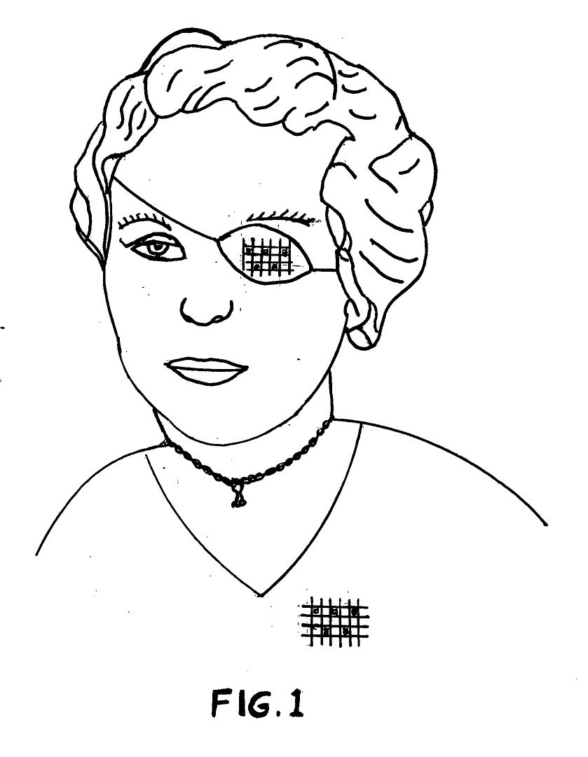

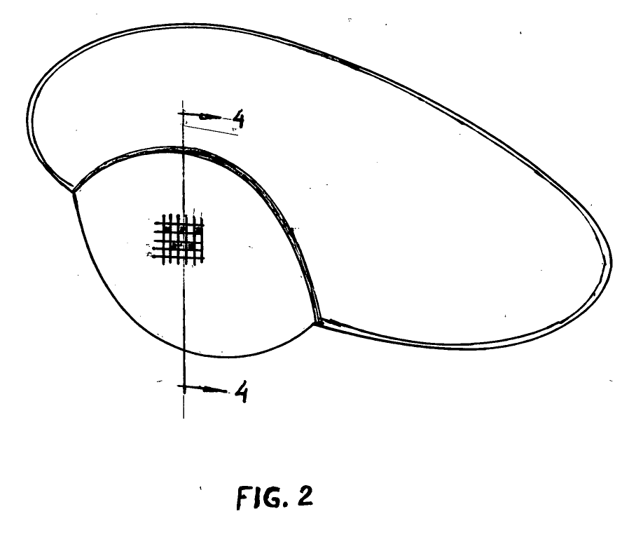

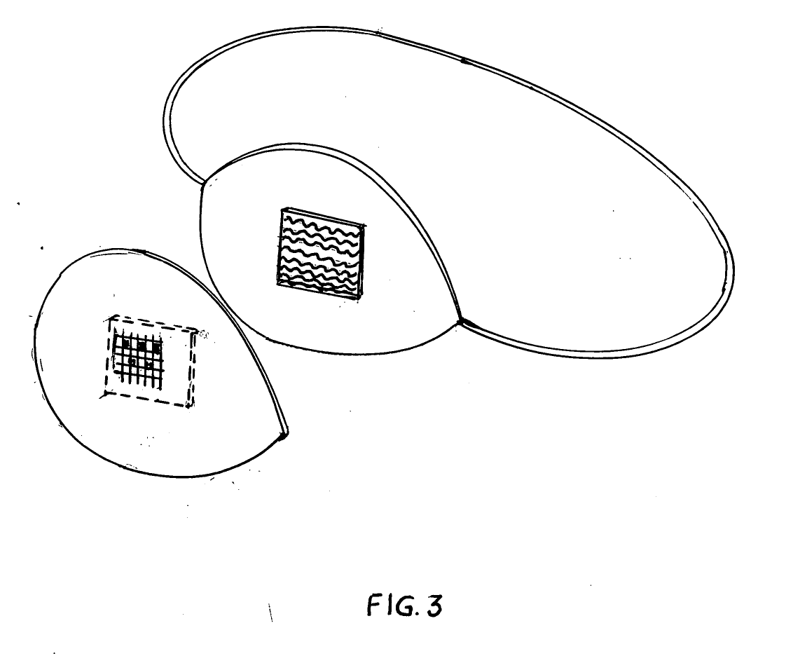

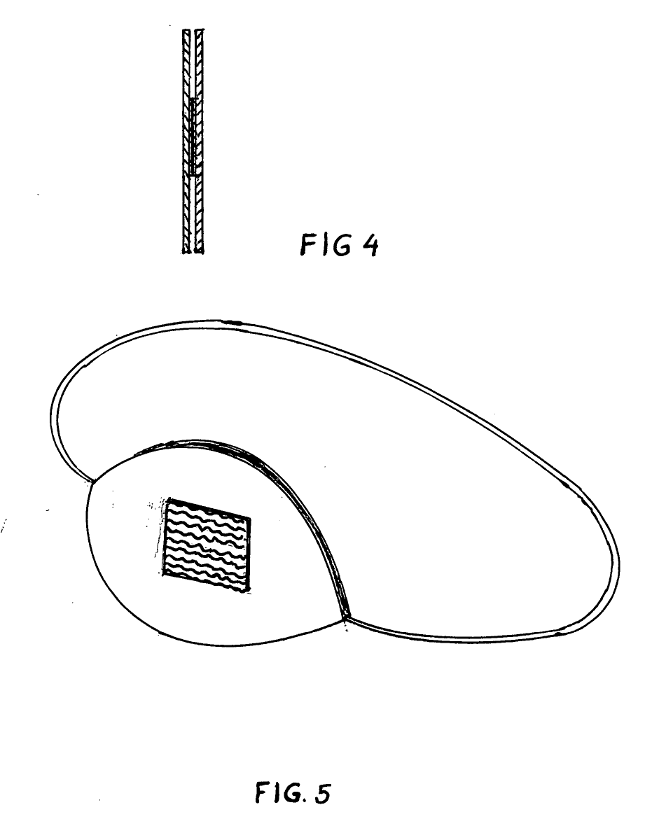

What is claimed in this invention:

1. The present invention provides an interchangeable decorative eye patch cover which can be used with commonly found store-bought eye patches.

2. The interchangeable decorative eye patch cover is shaped to conform to the shape of commonly found store-bought eye patches being sufficiently flat to lie atop commonly found store-bought eye patches.

3. The interchangeable decorative eye patch cover has a Velcro strip on the back that allows it to be affixed on top of a commonly found store-bought eye patch cover.
